(19) United States
(12) Patent Application Publication
(10) Pub. No.: US 2004/0241134 A1
(43) Pub. Date: Dec. 2, 2004

(54) Colon cleansing compositions and methods of use therefor

(76) Inventor: Mary K. Adams, Scottsdale, AZ (US)

Correspondence Address: WEISS & MOY PC, 4204 NORTH BROWN AVENUE, SCOTTSDALE, AZ, 85251, US

(21) Appl. No.: 10/446,437

(22) Filed: May 28, 2003

(51) Int. Cl. A61K 31/74; A61K 47/32

(52) U.S. Cl. 424/78.1

(57) ABSTRACT

The present invention discloses colon cleansing compositions for treating chronic constipation. The colon cleansing compositions comprise mixtures of Calcium Polycarbophil and flaxseed powder and the mixtures are preferably in tablet form. A method for using the colon cleansing compositions for treating chronic constipation is disclosed. The preferred method is to orally masticate and swallow two tablets after each meal and two tablets before bedtime, followed by drinking a sufficient quantity of fluid to activate the colon cleansing compositions in a person's digestive system leading to colon cleansing in a period of less than 72 hours. After the initial colon cleansing, a dosage of the colon cleansing composition is preferably orally masticated and swallowed at least one of: after breakfast, after dinner, and before bedtime; followed by drinking a sufficient quantity of fluid to prevent further constipation.

### FIELD OF THE INVENTION

This invention relates generally to constipation and methods for relieving constipation and more specifically to improved colon cleansing compositions and methods of use thereof for maintaining long term relief from chronic constipation.

### BACKGROUND OF THE INVENTION

Constipation is a digestive disorder that may lead to depression and fatigue, and may also be responsible for serious diseases of the colon. According to a number of opinions, constipation occurs when bowel movements of from three times a day to three times a week result in either no stool or small amounts of hard dry stool and/or painful elimination. A bowel movement that results in hard dry stool with straining during elimination causes rougher movement through the digestive system that leads to numerous side effects, including hemorrhoids. Almost all people will be constipated at some time in their lives, some more severely than others. People are more prone to suffer from symptoms of constipation as they age. Chronic constipation is a more severe form of constipation, which has greater debilitating effects on people suffering with this condition.

Constipation causes food transit times to decrease, resulting in any undigested waste matter that is retained in the digestive system for excessive periods of time to chemically breakdown undesirably and enter the bloodstream through the intestinal walls. Some of these waste matter breakdown products are thought to be toxins to the body, leading to symptoms such as headaches and autoimmune disorders. The build-up of toxins is thought to reduce the absorption of desirable nutrients (also known as malabsorption), thereby depriving the body of nutrients needed for energy and vitality as manifested by complaints of fatigue and depression. Some other conditions that may result from constipation include obesity, diverticulosis, PMS, bad breath, body odor, indigestion, gas, bloating, insomnia, arthritis and colon cancer.

In most cases, constipation is thought to arise from insufficient amounts of fiber and fluid in the diet. Other possible causes include inadequate exercise, advanced age, muscle disorders, a poor diet especially consisting of a high intake of junk foods, and side effects of other serious medical disorders. Constipation may also be a side effect of drugs, such as painkillers and antidepressants, as well as iron supplements.

In the past, numerous compositions and methods were suggested and used for relieving constipation. These ranged from anecdotal compositions such as high fiber vegetables and fruits, avoidance of certain types of foods and exercise routines, to the use of medicinally active compositions that provided some relief from constipation. Often, the composition or method would result in relief for a short period of time, but the regimen necessary to maintain long term relief from chronic constipation was not readily adopted.

In many cases of mild constipation, simple measures are adequate for relieving constipation. Drinking at least eight 8 ounce glasses of water is important to provide sufficient fluid content when fiber is added to the diet. Laxative juices and foods, such as prune juice or figs also are helpful and improve fluid intake. Sufficient fluid intake is important because constipation may result from dehydration. Fluids aid in making peristaltic action of the bowel more regular. Peristalsis is the natural wavelike movement of the colon that occurs during digestion of food. Lack of sufficient fluid disrupts proper peristalsis. Minerals such as milk of magnesia and Epsom salts, as well as herbs such as cascara, sagrada and senna stimulate peristaltic action by irritating the colon, causing it to expel its contents. These products are habit forming and therefore undesirable for long term resolution of bowel movement irregularity.

Sufficient fiber intake is another important parameter in relieving constipation. Fiber consists largely of digestible cellulose and other cell-wall polymers of plant origin that are not digested. Although fiber is not metabolized and is not digested, it aids proper motility of the intestinal tract. It has been suggested that fiber intake of about 30 to 40 grams per day is optimal. For many people, this amount of fiber intake is difficult to maintain. As a result, fiber supplements are added in the daily diet in the form of non-dietary and non-drug bulk producing fibers such as methylcellulose or Psyllium seed (Plantago ovato). The primary chemical constituents of Psyllium include mucilage (arabinoxylan), aucubine, protein enzymes, xylose, galactose, starch and linoleic, oleic and palmitic oils.

Flaxseed (Linum vistatissimum L., Linaceae) based fiber is preferred to Psyllium because Psyllium is almost completely soluble in water resulting in colon dehydration which reduces peristalsis. Flaxseed contains omega-6 and omega-9 essential fatty acids, linolenic, linoleic and oleic acids, linamarin (a cyanogenic glycoside), mucilage, cyanogenic glycosides, B vitamins, fiber, protein, potassium, lecithin, magnesium, zinc, a n d vitamins A and E. Flaxseed has three major components making it especially beneficial in human nutrition: (a) a very high content of essential fatty acid, alpha linoleic acid (omega-3 fatty acid); (b) a high percentage of dietary fiber (approximately 50% insoluble in water); and (c) the highest content of plant lignans (thought to be anticarcinogenic) of all plant or seed products used for human consumption. Flaxseed is thought to absorb toxins, while creating proper bulk in the colon without dehydrating it. Flaxseed also acts as a lubricant for the colon.

Recently, a synthetic material, Polycarbophil has been used to promote bowel regularity, especially for chronic constipation, diverticulosis and irritable bowel syndrome. Polycarbophil and its Calcium salt (Calcium Polycarbophil, USP, CAS Number 0025987-55-7) are available in over-the-counter form. Polycarbophil is a generic name for a class of acrylic polymers known as Carbopol® resins, manufactured by Noveon, Inc. Carbopol® resins are cross-linked homopolymers of acrylic acid or cross-linked copolymers of acrylic acid with a minor acrylic comonomer. Carbopol® resins have molecular weights ranging from 740,000 to 4-5 million. Calcium Polycarbophil is characterized as being an off-white powder, with a pH value of 6.0-8.0@1% in water, less than 10% volatile by weight consisting of water, and bulk density of 0.86 g/ml. Calcium Polycarbophil absorbs sixty times its weight in water and is not absorbed into the body. Its main function is to absorb water and expand to provide increased bulk and moisture content to soften the stool. Used by itself Calcium Polycarbophil has the negative characteristics of Psyllium, i.e. it results in colon dehydration which may reduce peristalsis unless sufficient water is taken during its use.

For the foregoing reasons, a need exists for improved compositions for relieving chronic constipation and methods of using such compositions. Such compositions must be safe and effective for human use and their method of use should preferably be easy to understand, routine and readily adopted. In addition, any side effects should be minimal, and additional benefits besides relieving the effects of chronic constipation are desirable. The present invention provides a combination of ingredients and methods suitable for meeting these needs.

### SUMMARY OF THE INVENTION

It is an object of the present invention to provide improved colon cleansing compositions.

It is a further object of the present invention to provide improved colon cleansing compositions suitable for treating the symptoms of chronic constipation.

It is a still further object of the present invention to provide improved colon cleansing compositions suitable for maintaining relief from chronic constipation.

It is a yet further object of the present invention to provide methods for using improved colon cleansing compositions suitable for treating and maintaining relief from chronic constipation.

### BRIEF DESCRIPTION OF THE PREFERRED EMBODIMENTS

In accordance with one embodiment of the present invention, a colon cleansing composition for treatment of chronic constipation is disclosed. The composition comprises in combination a Polycarbophil for absorbing water to increase bulk and moisture content in stool; and flaxseed powder for providing both soluble and insoluble fiber to the colon cleansing composition, the colon cleansing composition being substantially activated in a person's digestive system.

In accordance with another embodiment of the present invention, a method for treatment of chronic constipation is disclosed. The method comprises the steps of providing a colon cleansing composition comprising a Polycarbophil for absorbing water to increase bulk and moisture content in stool; and flaxseed powder for providing both soluble and insoluble fiber to the colon cleansing composition; and substantially activating the colon cleansing composition in a person's digestive system.

The foregoing and other objects, features, and advantages of the invention will be apparent from the following, more particular, description of the preferred embodiments of the invention.

### DETAILED DESCRIPTION OF THE PREFERRED EMBODIMENTS

The compositions of the present invention comprise a mixture of flaxseed powder and a Polycarbophil as active ingredients, thereby taking advantage of the physiological and chemical properties of both of these materials in combination. As discussed above, flaxseeds have many advantages including their ability to help relieve constipation and also provide nutrients that are thought to be beneficial to people. Flaxseeds are readily available from natural sources and are known to be safe for human ingestion. Flaxseed powder provides the further characteristic that it will not dehydrate the intestine during use, so that peristalsis occurs naturally when the composition is ingested with a fluid source such as water. A Polycarbophil is also safe for human ingestion and provides the bulk forming characteristics necessary to provide for moister stools on elimination.

The compositions of the present invention are preferably made up in chewable tablet form for facile use. However, chewable capsules, liquid formulations or powder compositions available in prepackaged or unpackaged form are also effective. Tablets, chewable capsules and prepackaged powder compositions allow delivery of pre-measured quantities of ingredients. Liquid formulations would be useful when a person has difficulty in swallowing a solid or semi-solid food. Liquid formulations could be administered intravenously directly into the digestive system.

Sucrose, citric acid, stearic acid, silicon dioxide and magnesium stearate are inactive ingredients that are included in a preferred tablet form of the composition. The inactive ingredients act as flavorings, preservatives, fillers and binding agents. It is to be understood that other inactive ingredients and combinations thereof may be used together with the active ingredients with equivalent effectiveness.

Flaxseed powder that is used in the compositions is preferably in the form of high lignin golden flaxseed powder. Other grades of flaxseed powder are less preferable, but would be efficacious. A preferable Polycarbophil is Calcium Polycarbophil, although it should be clearly understood that there is substantial benefit from an alternative formulation of the colon cleansing composition in which a different salt form of Polycarbophil is used.

While there are numerous mix ratios of flaxseed powder and a Polycarbophil that are useful for relieving chronic constipation, a colon cleansing composition preferably comprises 625 mg of Calcium Polycarbophil (comprising 125 mg Calcium cations and 500 mg of Polycarbophil anions) and 1000 mg of flaxseed powder. A tablet weighing 2800 mg formed from a preferable composition has 1175 mg of inactive ingredients.

Another composition that is less preferable, but which would still be effective, would have a lower limit of 250 mg of flaxseed powder together with a lower limit of 250 mg of Calcium Polycarbophil to make a smaller tablet with inactive ingredients as listed above. Ratios of flaxseed powder to Calcium Polycarbophil that exceed the lower individual limits of flaxseed powder and Calcium Polycarbophil, but do not exceed the upper limits of 1000 mg of flaxseed powder and 625 mg of Calcium Polycarbophil are less preferred but would also be effective.

A preferred method for using the preferred composition in tablet form to relieve chronic constipation for adults is to thoroughly chew and swallow two tablets followed by drinking at least 8 fluid ounces of a digestible fluid, preferably water, after each one of the meals breakfast, lunch and dinner, and two tablets before bedtime. After a period of 48 to 72 hours, colon cleansing results. Thereafter, for people requiring further maintenance, thoroughly chewing and swallowing two tablets of the preferred composition after breakfast and 2 tablets of the preferred composition after dinner, each followed by drinking at least 8 fluid ounces of water or some other digestible fluid, is adequate for maintaining relief from constipation. For less severe constipation, thoroughly chewing one or two tablets of the preferred composition followed by drinking at least 8 fluid ounces of water prior to bedtime is adequate. A preferred method for treating children older than six years of age and younger than twelve years of age uses half the quantities described above for adult relief of chronic constipation.

In an alternative method for using the preferred composition, tablets of the preferred composition pre-dissolved in a semi-fluid food, such as apple sauce, to form a mixture of the tablets and semi-fluid food are eaten followed by drinking at least 8 fluid ounces of water using the same colon cleansing method as described above. This alternative method provides for people who have difficulty in chewing and also insures that the tablets are thoroughly broken up before swallowing them.

It will be appreciated that methods of using other compositions as described above would follow similar guidelines as those for the preferred composition. For people suffering less severe constipation, smaller tablets would provide the needed quantities of active ingredients. Additionally, a colon cleansing following the preferred composition and method may be followed by using other lower active ingredient compositions as described above.

It is preferred, that a patient younger than six years of age, or a pregnant or nursing patient be monitored by a physician. In addition, monitoring by a physician is preferred when abdominal pain, nausea or vomiting are experienced by a patient. The composition of the invention is not to be used if taking a prescription antibiotic drug containing any form of a tetracycline.

In summary, the present invention is directed to colon cleansing compositions for treating chronic constipation. The colon cleansing compositions comprise mixtures of a Polycarbophil and flaxseed powder. The colon cleansing compositions are preferably in tablet form. A method for using the colon cleansing compositions for treating chronic constipation is disclosed. The preferred method is to orally masticate and swallow a dosage of the colon cleansing composition after each meal and before bedtime, followed by drinking a sufficient quantity of fluid to activate the colon cleansing compositions in a person's digestive system leading to colon cleansing in a period of less than 72 hours. After the initial colon cleansing, the dosage of the colon cleansing composition is preferably orally masticated and swallowed after breakfast and dinner and before bedtime, followed by drinking a sufficient quantity of fluid to prevent further constipation. In the case of less severe constipation, the dosage of the colon cleansing composition is preferably orally masticated and swallowed before bedtime, followed by drinking a sufficient quantity of fluid. An alternative method for using the colon cleansing compositions for treating chronic constipation is also disclosed. The alternative method is to add and mix a dosage of the colon cleansing composition into a semi-fluid food and swallow the mixture after each meal and bedtime, followed by drinking a sufficient quantity of fluid to activate the colon cleansing compositions in a person's digestive system leading to colon cleansing in a period of less than 72 hours. The alternative method follows the same procedure as the oral masticating method in all other respects. The dosages listed for each of the methods disclosed is reduced by half for children that are older than six years of age and younger than twelve years of age.

While the invention has been particularly shown and described with reference to preferred embodiments thereof, it will be understood by those skilled in the art that the foregoing and other changes in form and details may be made therein without departing from the spirit and scope of the invention. For example, the inactive ingredients may be altered to suit different dietary needs, such as the use of non-sucrose sweeteners for diabetics. The methods of using the composition of the invention may be varied by a consumer depending on the changing needs of the consumer.

What is claimed is:

1. A colon cleansing composition for treatment of chronic constipation comprising, in combination:
a Polycarbophil for absorbing water to increase bulk and moisture content in stool; and
flaxseed powder for providing both soluble and insoluble fiber to said colon cleansing composition, said colon cleansing composition being substantially activated in a person's digestive system.

2. The colon cleansing composition for treatment of chronic constipation according to claim 1 wherein said flaxseed powder is substantially a high lignin golden flaxseed powder.

3. The colon cleansing composition for treatment of chronic constipation according to claim 1 wherein said Polycarbophil is a salt of Polycarbophil.

4. The colon cleansing composition for treatment of chronic constipation according to claim 3 wherein said salt of Polycarbophil is Calcium Polycarbophil.

5. The colon cleansing composition for treatment of chronic constipation according to claim 4 wherein the weight ratio of Polycarbophil anions to Calcium cations of Calcium Polycarbophil is at least 4.

6. The colon cleansing composition for treatment of chronic constipation according to claim 4 further comprising a mixture of at least 250 mg and at most 625 mg by weight of Calcium Polycarbophil; and at least 250 mg and at most 1000 mg by weight of flaxseed powder.

7. The colon cleansing composition for treatment of chronic constipation according to claim 1 further comprising ingredients selected from the group consisting of sucrose, citric acid, stearic acid, silicon dioxide and magnesium stearate.

8. The colon cleansing composition for treatment of chronic constipation according to claim 1 wherein said colon cleansing composition comprising a form selected from the group consisting of a tablet, a capsule, a liquid formulation and a powder.

9. A method for treatment of chronic constipation comprising the steps of:
providing a colon cleansing composition comprising a Polycarbophil for absorbing water to increase bulk and moisture content in stool;
providing flaxseed powder for providing both soluble and insoluble fiber to said colon cleansing composition; and
substantially activating said colon cleansing composition in a person's digestive system.

10. The method for treatment of chronic constipation according to claim 9 wherein said flaxseed powder being substantially a high lignin golden flaxseed powder.

11. The method for treatment of chronic constipation according to claim 9 further comprising the steps of:
orally masticating said colon cleansing composition;
swallowing said masticated colon cleansing composition; and
drinking a sufficient quantity of a digestible fluid to substantially activate said colon cleansing composition in a person's digestive system.

12. The method for treatment of chronic constipation according to claim 11 further comprising the steps of:
as a means of continuing to relieve symptoms of constipation, providing a dosage of said colon cleansing composition after each meal and before bedtime, resulting in colon cleansing after a period of less than 72 hours; and
reducing the dosage of said colon cleansing composition after breakfast and after dinner so as to have regular bowel movements after completion of colon cleansing.

13. The method for treatment of chronic constipation according to claim 12 further comprising the steps of:
as a means of continuing to relieve symptoms of constipation, further reducing the dosage of said colon cleansing composition to none after both breakfast and dinner; and
consuming the dosage of said colon cleansing composition before bedtime after completion of colon cleansing so as to have regular bowel movements when the symptoms of constipation are less severe.

14. The method for treatment of chronic constipation according to claim 9 further comprising the step of reducing a dosage of said colon cleansing composition by one half when said treatment is for a child older than six years of age and younger than twelve years of age.

15. The method for treatment of chronic constipation according to claim 9 further comprising the steps of:
adding said colon cleansing composition to a semi-fluid food;
mixing said colon cleansing composition and said semi- fluid food;
swallowing a mixture of said colon cleansing composition and said semi-fluid food; and
drinking a sufficient quantity of a digestible fluid to substantially activate said colon cleansing composition in a person's digestive system.

16. The method for treatment of chronic constipation according to claim 15 further comprising the steps of:
as a means of continuing to relieve symptoms of constipation, providing a dosage of said colon cleansing composition after each meal and before bedtime, resulting in completion of colon cleansing after a period of less than 72 hours; and
reducing the dosage of said colon cleansing composition after breakfast and after dinner so as to have regular bowel movements after completion of colon cleansing.

17. The method for treatment of chronic constipation according to claim 16 further comprising the steps of:
as a means of continuing. to relieve symptoms of constipation, further reducing the dosage of said colon cleansing composition to none after both breakfast and dinner; and
consuming the dosage of said colon cleansing composition before bedtime after completion of colon cleansing so as to have regular bowel movements when the symptoms of constipation are less severe.

18. The method for treatment of chronic constipation according to claim 9 wherein said Polycarbophil being a salt of Polycarbophil.

19. The method for treatment of chronic constipation according to claim 18 wherein said salt of Polycarbophil being Calcium Polycarbophil.

20. The method for treatment of chronic constipation according to claim 19 wherein said colon cleansing composition comprising at least 250 mg and at most 625 mg by weight of Calcium Polycarbophil and at least 250 mg and at most 1000 mg by weight of flaxseed powder.

21. The method for treatment of chronic constipation according to claim 9 wherein said colon cleansing composition comprises ingredients selected from the group consisting of sucrose, citric acid, stearic acid, silicon dioxide and magnesium stearate.

22. The method for treatment of chronic constipation according to claim 9 further comprising the steps of:
providing said colon cleansing composition in a form selected from the group consisting of a tablet, a capsule, a liquid formulation and a powder;
providing at least 8 fluid ounces of a digestible fluid after consuming said colon cleansing composition; and
consuming said colon cleansing composition; and
consuming a sufficient quantity of said digestible fluid to substantially activate said colon cleansing composition in a person's digestive system.
